(19) United States
(12) Patent Application Publication
(10) Pub. No.: US 2011/0306904 A1
(43) Pub. Date: Dec. 15, 2011

(54) ABLATION DEVICES AND RELATED METHODS THEREOF

(75) Inventors: Jason Jacobson, Chicago, IL (US); Jason Rubenstein, Brookfield, WI (US); Michael Kim, Wilmette, IL (US)

(73) Assignee: NORTHWESTERN UNIVERSITY, Evanston, IL (US)

(21) Appl. No.: 13/060,632

(22) PCT Filed: Aug. 25, 2009
(86) PCT No.: PCT/US2009/054912

§ 371 Date: May 11, 2011

Related U.S. Application Data
(60) Provisional application No. 61/091,837, filed on Aug. 26, 2008.

(51) Int. Cl. A61B 18/00; A61B 18/02; A61B 18/18

(52) U.S. Cl. 601/2; 606/1; 606/33; 606/20

(57) ABSTRACT

The present invention relates generally to devices for performing targeted tissue ablation in a subject. In particular, the present invention provides devices configured to deliver energy to a targeted tissue region without causing damage to untargeted tissue.

### CROSS-REFERENCE TO RELATED APPLICATIONS

The present application claims the benefit of pending Provisional patent application No. 61/091,837, filed Aug. 26, 2008, which is hereby incorporated by reference in its entirety.

### FIELD OF THE INVENTION

The present invention relates generally to methods and devices for performing targeted tissue ablation in a subject. In particular, the present invention provides devices configured to deliver energy to a targeted tissue region without causing damage to untargeted tissue.

### BACKGROUND OF THE INVENTION

Radiofrequency energy is used to destroy abnormal electrical pathways in, for example, heart tissue. It is used in recurrent atrial fibrillation and other types of supraventricular tachycardia. In practice, an energy emitting probe (electrode) is placed into the heart through a catheter. The practitioner first “maps” an area of the heart to locate the abnormal electrical activity before the responsible tissue is eliminated.

However, damage (e.g., undesired thermal injury) to tissue regions that are in contact with the ablated tissue during an ablation procedure can lead to severe complications, and even death. Avoidance of these risks has limited the location and nature of ablative treatments, limiting options for physicians and patients. As such, improved ablation devices and methods are needed.

### SUMMARY OF THE INVENTION

The present invention relates generally to methods and devices for performing targeted tissue ablation in a subject. In particular, the present invention provides devices configured to deliver energy to a targeted tissue region without causing damage to untargeted tissue.

In certain embodiments, the present invention provides devices configured to ablate a targeted tissue region while preventing thermal damage to surrounding tissue. The devices are not limited to ablating a particular targeted tissue region. In some embodiments, the targeted tissue region is within the pericardial space. In some embodiments, the devices may be utilized in treating cardiac disorders including, but not limited to, atrial fibrillation, multifocal atrial tachycardia, inappropriate sinus tachycardia, atrial tachycardia, ventricular tachycardia, ventricular tachycardia, and Wolff-Parkinson-White syndrome.

In some embodiments, the devices comprise an elongate catheter body and a deployable procedure region. In some embodiments, the deployable procedure region is configured to deliver ablative energy to a targeted tissue region while protecting non-targeted tissue regions from thermal injury. In some embodiments, the deployable procedure region has therein an ablative region and a thermoprotective region. In some embodiments, the deployable procedure region has a shape selected from the group consisting of a balloon shape and a sail shape, although the invention is not limited to these shapes. In some embodiments, the ablative region is designed to contact tissue targeted for ablation. In some embodiments, the thermoprotective region is designed to prevent thermal injury to non-targeted tissue regions. In some embodiments, the ablative region has thereon at least one electrode. In some embodiments, the deployable procedure region is configured to assume a deployed position and a non-deployed position.

The devices are not limited to delivering a particular type of energy. In some embodiments, the delivered energy is, for example, radio-frequency energy, microwave energy, cryo-energy energy, or ultrasound energy.

The elongate catheter body is not limited to a particular configuration and/or function. In some embodiments, the elongate catheter body is hollow. In some embodiments, the elongate catheter body is steerable. In some embodiments, the elongate catheter body has thereon at least one temperature probe. In some embodiments, the elongate catheter body is configured to circulate a fluid (e.g., saline) for purposes of reducing the temperature of the device.

In certain embodiments, the present invention provides methods for ablating a tissue region, comprising providing an ablation device of the present invention, and a subject having a tissue region requiring ablation (e.g., pericardial space) and a surrounding tissue region, positioning the device at the tissue region, deploying the deployable procedure region such that the ablative region is in contact with the tissue region and the thermoprotective region is in contact with the surrounding tissue region, and providing energy to the tissue region requiring ablation such that the surrounding tissue region is protected from thermal injury. In some embodiments, the tissue region requiring ablation is epicardial cardiac tissue. In some embodiments, the surrounding tissue region comprises esophageal tissue. In some embodiments, the surrounding tissue region comprises phrenic nerve tissue. In some embodiments, the devices may be utilized in treating cardiac disorders including, but not limited to, atrial fibrillation, multifocal atrial tachycardia, inappropriate sinus tachycardia, atrial tachycardia, ventricular tachycardia, ventricular tachycardia, and Wolff-Parkinson-White syndrome.

### DESCRIPTION OF THE FIGURES

FIG. 1 illustrates an ablation device embodiment including broadly an elongate catheter body and a deployable procedure region.

FIG. 2 presents an ablation device having an elongate catheter body and a deployable procedure region with an ablative region and a thermoprotective region behind the ablative region.

FIG. 3 presents an ablation device having an elongate catheter body and a balloon-shaped deployable procedure region with an ablative region and a thermoprotective region.

FIG. 4 presents an ablation device having an elongate catheter body and a deployable procedure region with an ablative region and a thermoprotective region behind the ablative region.

FIG. 5 presents an ablation device having an elongate catheter body and a sail-shaped deployable procedure region with an ablative region and a thermoprotective region behind the ablative region.

FIG. 6 presents a side view of an ablation device having an elongate catheter body and a sail-shaped deployable procedure region with an ablative region and a thermoprotective region behind the ablative region.

### DEFINITIONS

To facilitate an understanding of the invention, a number of terms are defined below.

As used herein, the terms “subject” and “patient” refer to any animal, such as a mammal like livestock, pets, and preferably a human. Specific examples of “subjects” and “patients” include, but are not limited, to individuals requiring medical assistance, and in particular, requiring catheter ablation treatment.

As used herein, the terms “catheter ablation” or “ablation procedures” or “ablation therapy,” and like terms, refer to what is generally known as tissue destruction procedures. Ablation is often used in treating several medical conditions, including abnormal heart rhythms.

As used herein, the term “energy” or “energy source,” and like terms, refers to the type of energy utilized in ablation procedures. Examples include, but are not limited to, radio-frequency energy, microwave energy, cryo-energy energy (e.g., liquid nitrogen), and ultrasound energy.

### DETAILED DESCRIPTION OF THE INVENTION

The normal functioning of the heart relies on proper electrical impulse generation and transmission. In certain heart diseases (e.g., atrial fibrillation) proper electrical generation and transmission are disrupted. In order to restore proper electrical impulse generation and transmission, catheter ablation therapies may be employed.

In general, catheter ablation therapy provides a method of treating tissues having, for example, electrical impulse dysfunction (e.g., cardiac arrhythmias). Physicians make use of catheters to gain access into interior regions of the body. Catheters with attached ablating devices are used to destroy targeted tissue. In the treatment of cardiac arrhythmias, a specific area of cardiac tissue emitting or conducting erratic electrical impulses is initially localized. A user (e.g., a physician) will direct a catheter through a main vein or artery into the interior region of the heart that is to be treated. The ablating element is next placed near the targeted cardiac tissue that is to be ablated. The physician directs an energy source from the ablating element to ablate the tissue and form a lesion.

In general, the goal of catheter ablation therapy is to destroy tissue (e.g., cardiac tissue) suspected of emitting erratic electric impulses, thereby curing the tissue (e.g., heart tissue) of the dysfunction. One problem associated with electrophysiology ablation procedures involves undesired thermal injury of non-targeted tissue regions (e.g., tissue regions surrounding the targeted tissue region). For example, during invasive electrophysiology ablations, damage to surrounding extra-cardiac structures is at risk for thermal injury when the underlying myocardium is heated during intracardiac RF lesion delivery. Such undesired thermal injury damage results, for example, from radiated thermal energy from heating nearby tissue, and from direct RF heating to the extracardiac tissue. The devices of the present invention overcome these limitations. In particular, the devices of the present invention are configured to perform targeted tissue ablation while preventing undesired thermal injury of non-targeted tissue.

The present invention also provides tissue ablation systems, and methods for using such ablation systems. The exemplary embodiments embodiments discussed in more detail below illustrate use of the devices for catheter-based cardiac ablation. These structures, systems, and techniques are well suited for use in the field of cardiac ablation. However, it should be appreciated that the invention is applicable for use in other tissue ablation applications. For example, the various aspects of the invention have application in procedures for ablating tissue in the prostrate, brain, gall bladder, uterus, and other regions of the body, using systems that are not necessarily catheter-based.

In some embodiments, the devices of the present invention have a deployable procedure region having one surface configured to deliver energy to a tissue (e.g., via an electrode array) and a second surface having a thermoprotective coating. In some embodiments, the shape of the deployable procedure region, when deployed, is configured to match a tissue region targeted for ablation (e.g., configured to match left or right pulmonary vein recesses). Such a shape may be achieved with a balloon structure, a sail-type structure, or other approaches.

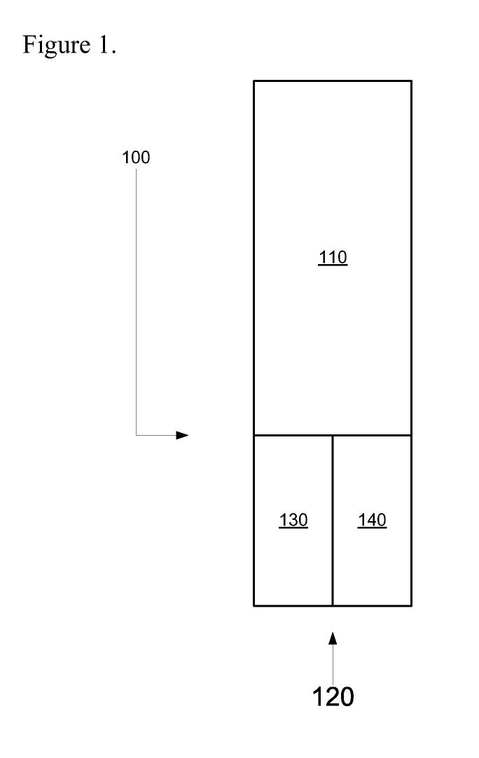

In some embodiments, the present invention provides balloon-type ablation devices configured to perform targeted tissue ablation while preventing undesired thermal injury of non-targeted tissue. In some embodiments, the present invention provides sail-type ablation devices configured to perform targeted tissue ablation while preventing undesired thermal injury of non-targeted tissue. FIGS. 1-6 shows various embodiments of the balloon-type ablation devices and sail-type ablation devices of the present invention. The present invention is not limited to these particular configurations.

FIG. 1 illustrates an ablation device 100 embodiment including broadly an elongate catheter body 110 and a deployable procedure region 120. The ablation device 100 is not limited to a particular shape and/or configuration. In some embodiments, the ablation device 100 is configured to perform targeted tissue ablation (e.g., cardiac tissue ablation) while preventing undesired thermal injury of non-targeted tissue (e.g., non-targeted cardiac tissue). The ablation device 100 is not limited to delivering a particular type of energy (e.g., radio-frequency energy, microwave energy, cryo-energy energy (e.g., liquid nitrogen), or ultrasound energy). In addition, the ablative device 100 is configured to deliver energy to a tissue region in a controlled manner (e.g., continuous energy deliver, non-continuous energy deliver, timed energy delivery, etc.).

Still referring to FIG. 1, the elongate catheter body 110 is not limited to a particular shape or configuration. In some embodiments, the elongate catheter body 110 is configured to receive energy from an energy source, transmit the energy along its length, and deliver the energy to the deployable procedure region 120. The elongate catheter body 110 is not limited to receiving, transmitting, and delivering a particular kind of energy. In some embodiments, the elongate catheter body 110 receives, transmits and delivers, for example, radio-frequency energy, and/or microwave energy. In some embodiments, the elongate catheter body 110 is hollow. In some embodiments, the elongate catheter body 110 is steerable so as to permit navigation of the ablation device 100 (e.g., through a catheter; through a vein; through an artery; through an organ). The elongate catheter body 110 is not limited to particular size dimensions. In some embodiments, the elongate catheter body 110 ranges in size such that it is not so small that it cannot carry necessary ablation items, and not so large so that it cannot fit in a peripheral major vein or artery. In some embodiments, the elongate catheter body 110 includes an elongate sheath (e.g., protective covering). The elongate catheter body 110 is not limited to a particular material composition. In some embodiments, the elongate catheter body 110 is made of a polymeric, electrically nonconductive material, like polyethylene or polyurethane. In some embodiments, the elongate catheter body 110 is formed with the nylon based plastic Pbax, which is braided for strength and stability. In some embodiments, the elongate catheter body 110 is formed with hypo tubing (e.g., stainless steel, titanium).

Still referring to FIG. 1, in some embodiments, the elongate catheter body 110 is not limited to housing particular items. In some embodiments, the elongate catheter body 110 permits the housing of items that assist in the ablation of a subject's tissue (e.g., human tissue and other animal tissue, such as cows, pigs, cats, dogs, or any other mammal). In some embodiments, the elongate catheter body 110 houses, for example, a conducting wire (e.g., standard electrical wire), a steering device (e.g., a steering spring) (e.g., for purposes of navigating the ablation device 100), a thermal monitoring circuit (e.g., a temperature probe) (e.g., for purposes of monitoring the temperature of the ablation device 100, and providing such information to a user), a temperature regulation means (e.g., a saline exchange capability designed to control the temperature of the ablation device 100 and surrounding tissues, thereby permitting deeper ablation burns within a targeted tissue region). The present invention is not limited to a particular kind of thermal monitoring circuit. In some embodiments, the present invention utilizes a thermal monitoring circuit as described in U.S. Pat. No. 6,425,894 (herein incorporated by reference), whereby a thermocouple is comprised of a plurality of thermal monitoring circuits joined in series. The thermal monitoring circuits are thermoconductively coupled to the electrodes. In some embodiments, the thermal monitoring circuit employs two wires to travel through the elongate catheter body 110 in order to monitor a plurality of electrodes in, for example, the deployable procedure region 120 and/or along the length of the elongate catheter body 110. In some embodiments, the devices of the present invention utilize temperature monitoring systems. In some embodiments, temperature monitoring systems are used to monitor the temperature of an energy delivery device (e.g., with a temperature sensor). In some embodiments, temperature monitoring systems are used to monitor the temperature of a tissue region (e.g., tissue being treated, surrounding tissue). In some embodiments, the temperature monitoring systems are designed to communicate with a processor for purposes of providing temperature information to a user or to the processor to allow the processor to adjust the device appropriately.

Still referring to FIG. 1, the deployable procedure region 120 is configured to perform targeted tissue ablation (e.g., cardiac tissue ablation) while preventing undesired thermal injury of non-targeted tissue (e.g., non-targeted cardiac tissue). In some embodiments, the deployable procedure region 120 protects undesired thermal injury resulting from ablation induced from the same device. In some embodiments, the deployable procedure region 120 protects undesired thermal injury resulting from ablation induced from a different instrument (e.g., a separate endocardial ablation catheter). The deployable procedure region 120 is configured such that it can be presented in a closed position (e.g., non deployed state), open position (e.g., fully deployed state), or intermediate position (e.g., partially open and partially closed state). The deployable procedure region 120 is not limited to a particular shape. In some embodiments, the deployable tissue region 120 has thereon imaging markers (e.g., radioopaque markers) that indicate, for example, orientation of the ablative device 100 in a procedure (e.g., thereby ensuring the proper tissue is being ablated).

In some embodiments, the shape of the deployable procedure region 120 is a balloon shape. In some embodiments wherein the shape of the deployable procedure region 120 is of a balloon, the balloon is a standard inflatable percutaneous intervention balloon (e.g., a venoplasty balloon). In some embodiments wherein the deployable procedure region 120 is balloon shaped, the balloon is configured to adjust to the shape of a tissue region. In some embodiments wherein the deployable procedure region 120 is balloon shaped, the balloon may be partially or fully inflated or deflated. In some embodiments involving ablation of cardiac tissue, a pancake-shaped balloon that is wider than it is deep (e.g., 1.5× wider than deep; 2× wider than deep; 5× wider than deep; 10× wider than deep; 25× wider than deep) is used to provide protection to esophageal tissue (e.g., protection from thermal damage). In some embodiments involving ablation of cardiac tissue, a tall and narrow balloon (e.g., 1.5× taller than wide; 2× taller than wide; 3× taller than wide; 5× taller than wide; 10× taller than wide; 25× taller than wide) is used in the left or right pulmonary vein recesses to provide protection to the phrenic nerves (e.g., protection from thermal damage).

In some embodiments, the shape of the deployable procedure region 120 is a sail shape. In some embodiments wherein the deployable procedure region 120 is sail shaped, the deployable procedure region 120 is not limited to a particular number of sails (e.g., one sail, two sails, three sails, five sails, ten sails). In some embodiments wherein the deployable procedure region 120 is sail shaped, the sail is flat. In some embodiments wherein the deployable procedure region 120 is sail shaped, the sail is configured to adjust to the shape of a tissue region. In some embodiments wherein the deployable procedure region 120 is sail shaped, the sails may be partially and/or fully unfurled or furled. In some embodiments wherein the deployable procedure region 120 is sail shaped, the sails are rigid such that each sail has low to no flexibility. In some embodiments wherein the deployable procedure region 120 is sail shaped, the sails are non-rigid such that each sail has high flexibility (e.g., able to accommodate the shape of a tissue region).

Still referring to FIG. 1, the deployable procedure region 120 has therein an ablative region 130 and a thermoprotective region 140. In some embodiments, the ablative region 130 serves to provide energy to a tissue region (e.g., for purposes of ablating the tissue) while the thermoprotective region 140 serves to protect non-targeted tissue regions from thermal damage. The ablative region 130 and thermoprotective region 140 are not limited to particular size dimensions. In some embodiments, the size of the ablative region 130 is approximately half the size of the deployable procedure region 120 (e.g., 45%, 50%, 55%) and the size of the thermoprotective region 140 is approximately half the size of the deployable procedure region 120 (e.g., 45%, 50%, 55%). In some embodiments, the ratio of the sizes of the ablative region 130 and thermoprotective region 140 in relation to the deployable procedure region 120 can be, respectively, 10:1, 7.5:1, 5:1, 2.5:1, 1:1, 1:2.5, 1:5, 1:7.5, and 1:10. In some embodiments, the ratio of the sizes of the ablative region 130 and thermoprotective region 140 in relation to the deployable procedure region 120 is such that it maximizes the desired ablation procedure while protecting non-targeted tissue regions from thermal damage. As shown in FIG. 1, the ratio of the sizes of the ablative region 130 and thermoprotective region 140 in relation to the deployable procedure region 120 is 1:1.

Still referring to FIG. 1, the present invention is not limited to a particular type of ablative region 130. In some embodiments, the ablative region 130 is configured to deliver energy (e.g., radio-frequency energy, microwave energy, cryo-energy energy (e.g., liquid nitrogen), or ultrasound energy) from the ablation device 100 to a tissue region (e.g., cardiac tissue) (e.g., such that the tissue is ablated). In some embodiments, the ablative region 130 has therein an electrode layer (e.g., multi-conductor electrodes). In some embodiments, the ablative region 130 has therein an electrode layer positioned in a manner conducive for ablating a tissue region. The ablative region 130 is not limited to particular types of electrodes (e.g., platinum electrodes, copper electrodes, aluminum electrodes, etc.). In some embodiments, the electrodes report individual location impedences when not ablating (e.g., thereby assisting in determining good tissue contact (e.g., determining if the ablative region 130 is overlying pericardial fat or coronary arteries)). In some embodiments, by having multiple points to measure impedence from, it is possible to determine the position of a different impedence structure (e.g., coronary artery) in relation to the ablation device 100 (e.g., underneath the ablation device 100, on the right of the ablation device 100, on the left of the ablation device 100). In some embodiments, by having multiple points to measure impedence from, it is possible to confirm that the ablative region 130 is facing the targeted tissue region (e.g., epicardium). In some embodiments, the ablative region 130 has therein temperature sensors designed, for example, to continuously detect the temperature of a tissue region and provide such information to a user.

Still referring to FIG. 1, the present invention is not limited to a particular type of thermoprotective region 140. In some embodiments, the thermoprotective region 140 limits transmission of the thermal energy (e.g., radiant thermal energy from the ablative region 130) from a targeted tissue region (e.g., epicardial surface) to non-targeted tissue regions (e.g., the phrenic nerve, the esophagus, non-targeted cardiac tissue regions). In some embodiments, the thermoprotective region 140 is not limited to a particular material composition. In some embodiments, the thermoprotective region 140 is made of a polymeric, electrically nonconductive material, like polyethylene or polyurethane. In some embodiments, the thermoprotective region 140 is formed with the nylon based plastic Pbax, which is braided for strength and stability. In some embodiments, the thermoprotective region 140 is formed with a material having high insulating ability.

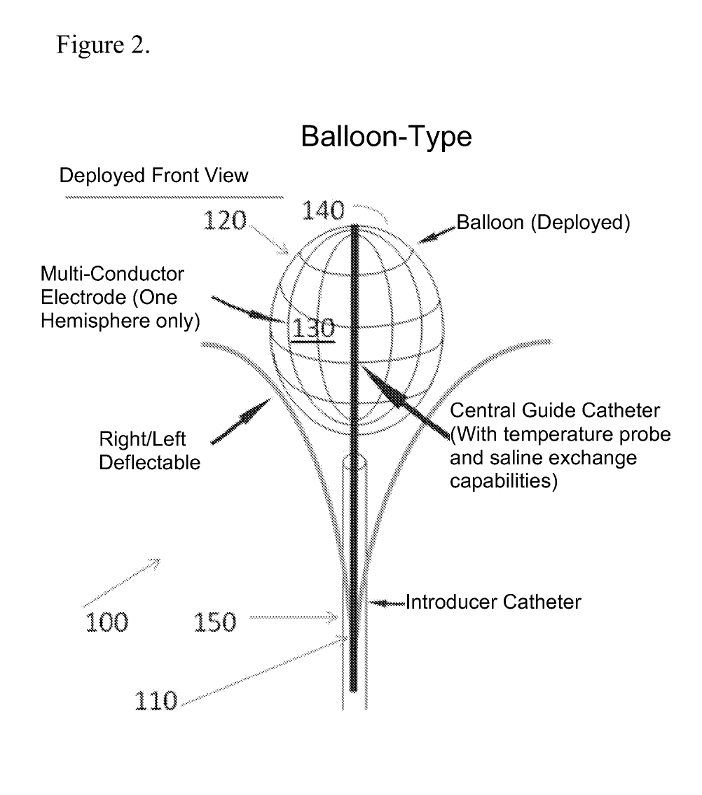

FIG. 2 presents an ablation device 100 having an elongate catheter body 110 and a deployable procedure region 120 with an ablative region 130 and a thermoprotective region 140 behind the ablative region 130. As shown, a portion of the elongate catheter body 110 is positioned within a catheter 150 (e.g., a catheter placed within a subject), and the deployable procedure region 120 is positioned beyond the terminus of the catheter in a deployed state. The shape of the deployable procedure region 120 is balloon-shaped, and the ablative region 130 has thereon a grid of electrodes designed to deliver energy to a targeted tissue. Such an embodiment permits the ablation of tissue in contact with the ablative region 130 while protecting non-targeted tissue from thermal injury (e.g., through inhibiting transmission of radiant energy with the thermoprotective region).

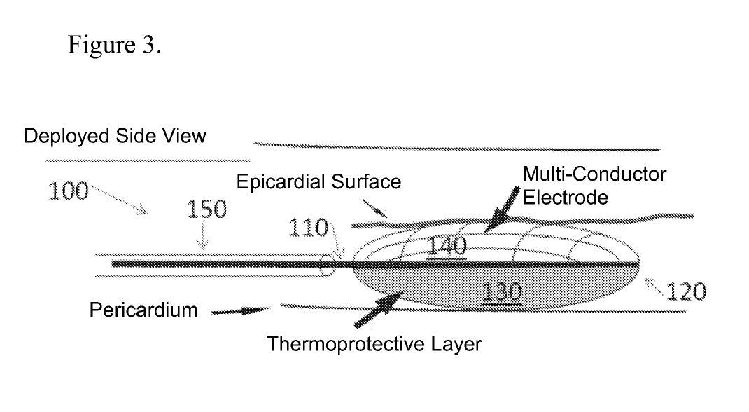

FIG. 3 presents an ablation device 100 having an elongate catheter body 110 and a balloon-shaped deployable procedure region 120 with an ablative region 130 (e.g., having a grid of electrodes) and a thermoprotective region 140. As shown, a portion of the elongate catheter body 110 is positioned within a catheter (e.g., a catheter placed within a subject), and the deployable procedure region 120 is positioned beyond the terminus of the catheter in a deployed state. In addition, as shown, the ablative region 130 is shown in contact with a targeted tissue region 160 (e.g., an epicardial surface) and the thermoprotective region 140 is shown in contact with a non-targeted tissue region 170 (e.g., pericardium). Such an embodiment permits the ablation of tissue in contact with the ablative region 130 while protecting non-targeted tissue from thermal injury (e.g., through inhibiting transmission of radiant energy with the thermoprotective region).

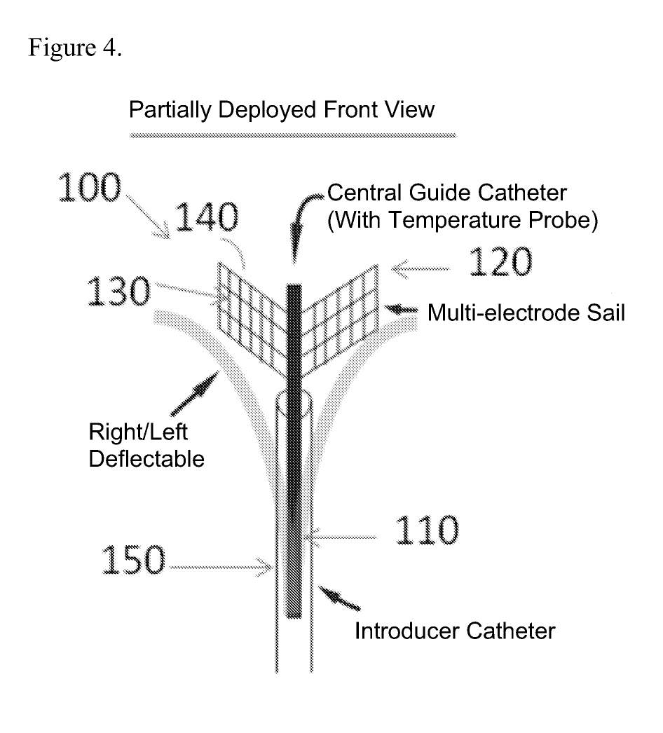

FIG. 4 presents an ablation device 100 having an elongate catheter body 110 and a deployable procedure region 120 with an ablative region 130 and a thermoprotective region 140 behind the ablative region 130. As shown, a portion of the elongate catheter body 110 is positioned within a catheter 150 (e.g., a catheter placed within a subject), and the deployable procedure region 120 is positioned beyond the terminus of the catheter in a deployed state. The shape of the deployable procedure region 120 is sail-shaped, and the ablative region 130 has thereon a grid of electrodes designed to deliver energy to a targeted tissue. As shown, the deployable procedure region 120 has therein two sail shaped regions. Such an embodiment permits the ablation of tissue in contact with the ablative region 130 while protecting non-targeted tissue from thermal injury (e.g., through inhibiting transmission of radiant energy with the thermoprotective region).

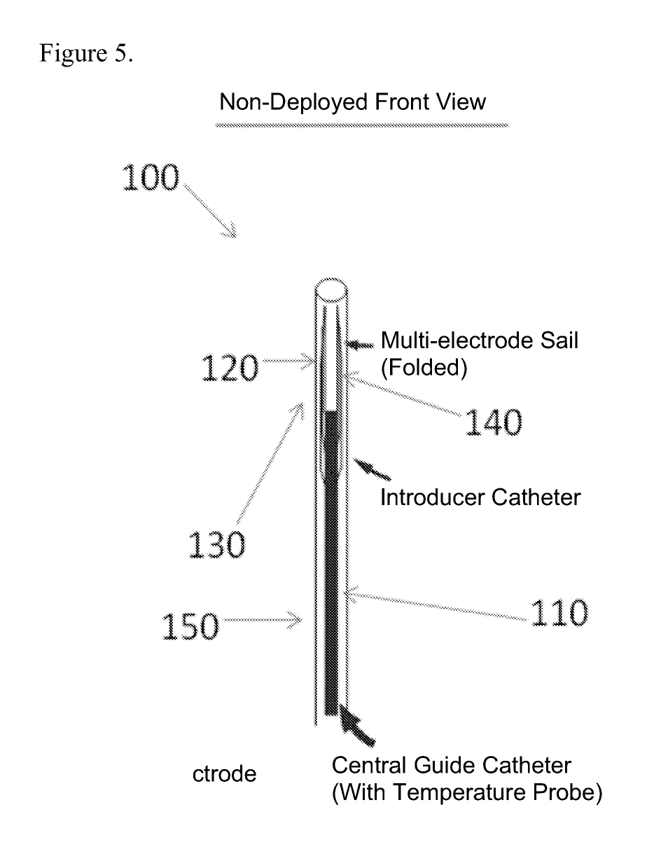

FIG. 5 presents an ablation device 100 having an elongate catheter body 110 and a sail-shaped deployable procedure region 120 with an ablative region 130 and a thermoprotective region 140 behind the ablative region 130. As shown, the elongate catheter body 110 is positioned within a catheter 150 (e.g., a catheter placed within a subject), and the deployable procedure region 120 is also positioned within the catheter 150 in a non-deployed state. Such an embodiment permits navigation of the ablation device 100 through narrow regions (e.g., catheters) without compromising the integrity of the deployable procedure region 120.

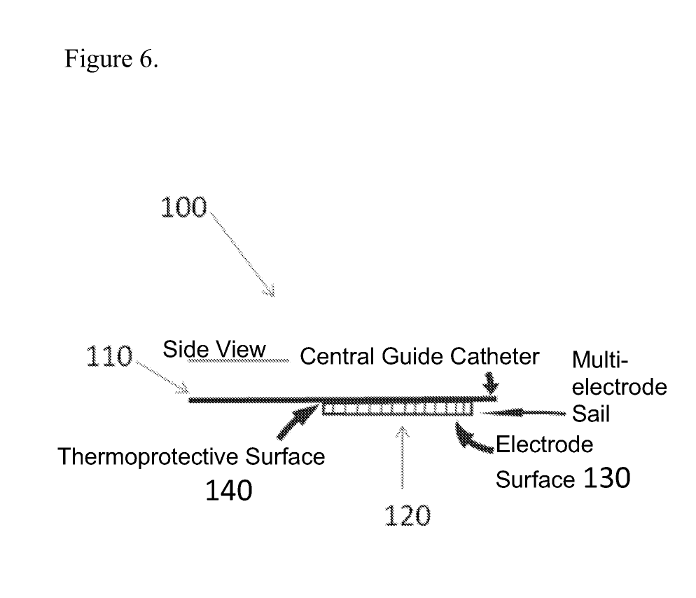

FIG. 6 presents a side view of an ablation device 100 having an elongate catheter body 110 and a sail-shaped (e.g., two sails) deployable procedure region 120 with an ablative region 130 and a thermoprotective region 140 behind the ablative region 130. As shown, the sail-shaped deployable procedure region 120 is presented in a deployed state, and the ablative region 130 has thereon a grid of electrodes designed to deliver energy to a targeted tissue. Such an embodiment permits the ablation of tissue in contact with the ablative region 130 while protecting non-targeted tissue from thermal injury (e.g., through inhibiting transmission of radiant energy with the thermoprotective region).

The ablation devices of the present invention are not limited to particular uses. For example, the ablation devices of the present invention find use in ablation procedures involving a high risk for damage to surrounding non-targeted tissue regions (e.g., avoiding phrenic nerve damage during epicardial ablation; avoiding esophageal damage during epicardial ablation).

The ablation devices of the present invention may be combined within various system embodiments. For example, the present invention provides systems comprising the ablation device along with any one or more accessory agents (e.g., catheters, sedation related drugs, imaging agents). The present invention is not limited to any particular accessory agent. Additionally, the present invention contemplates systems comprising instructions (e.g., surgical instructions, pharmaceutical instructions) along with the ablation devices of the present invention and/or a pharmaceutical agent (e.g., a cardiac medication). In some embodiments, the present invention provides systems utilizing one or more of the devices. In some embodiments, the systems provide devices having two or more (e.g., 2, 3, 5, 10) deployable procedure regions (e.g., using two or more catheters).

In some embodiments, the devices and systems are used with additional medical instruments (e.g., separate endocardial ablation catheters). In some embodiments, the devices are configured for use with additional medical instruments (e.g., a separate endocardial ablation catheter) so as to prevent undesired thermal injury resulting from the additional medical instrument.

In some embodiments, the devices and systems of the present invention utilize processors control one or more aspects of a device (e.g., deployment of the deployable procedure region; delivery of energy to a tissue region; relaying of tissue temperature information). In some embodiments, the processor is provided within a computer module. The computer module may also comprise software that is used by the processor to carry out one or more of its functions.

In some embodiments, the devices and systems of the present invention utilize imaging systems comprising imaging devices. The devices and systems are not limited to particular types of imaging devices (e.g., endoscopic devices, stereotactic computer assisted neurosurgical navigation devices, thermal sensor positioning systems, motion rate sensors, steering wire systems, and intraoperative magnetic resonance imaging). In some embodiments, the systems utilize endoscopic cameras, imaging components, and/or navigation systems that permit or assist in placement, positioning, and/or monitoring of any of the devices and systems of the present invention.

In some embodiments, the devices and systems provide software configured for use of imaging equipment (e.g., CT, MRI, ultrasound). In some embodiments, the imaging equipment software allows a user to make predictions based upon known thermodynamic and electrical properties of tissue and location of a device. In some embodiments, the imaging software allows the generation of a three-dimensional map of the location of a tissue region (e.g., a heart tissue region), location of the device(s), and to generate a predicted map of the ablation zone.

In some embodiments, the devices and systems are configured for percutaneous, intravascular, intracardiac, laparoscopic, or surgical delivery of energy. In some embodiments, the devices and systems are configured for delivery of energy to a target tissue or region while protecting surrounding tissue regions from thermal injury. The present invention is not limited by the nature of the target tissue or region. In some embodiments, the devices of the present invention may be utilized in treating cardiac disorders (e.g., cardiac disorders within the pericardial space) including, but not limited to, atrial fibrillation, multifocal atrial tachycardia, inappropriate sinus tachycardia, atrial tachycardia, ventricular tachycardia, ventricular tachycardia, and Wolff-Parkinson-White syndrome. In addition, the ablation devices of the present invention may be utilized in several other medical treatments (e.g., ablation of solid tumors, destruction of tissues, assistance in surgical procedures, kidney stone removal, etc.).

### EXAMPLE

This example describes an exemplary method for ablating cardiac tissue while protecting the esophageal thermal damage. While this example describes the ablation of cardiac tissue while protecting esophageal tissue from thermal damage, the technique may be applied to any tissue region. Generally, an ablation device is placed into the pericardial space via percutaneous pericardial access and maneuvered to the area overlying the site of desired ablation. The shape and size of the ablation device will be specific for use within the pericardial space. An ablation device having a balloon shaped (e.g., pancake-shaped balloon) deployable procedure region that is wider than it is deep will fit into the oblique sinus thereby providing esophageal protection. Then the active portion of the ablation device is deployed. The deployable tissue region consists of two surfaces: a thermoprotective region and an ablative region. The ablative region faces the epicardium and contains a metal electrode to serve as the ablation indifferent electrode. The thermoprotective region is positioned on the surface facing away from the myocardium and towards the visceral pericardial surface (e.g., towards the phrenic nerve). The ablative region (e.g., having electrodes) serves as the ablation indifferent electrode, and thereby prevents energy delivery to tissue beyond the myocardium, thereby reducing direct energy delivery to the non-cardiac tissues. Indeed, often, RF ablation lesions delivered from an endocardial catheter to a body-surface grounding pad will not have the energy delivery necessary for a deep myocardial burn without causing too high of blood-pool temperatures. By applying the ablation devices to the epicardial surface adjacent to the endocardial ablation catheter, the indifferent electrode focuses the energy to the myocardium only, allowing for deeper tissue lesions without high temperatures. In addition, the thermoprotective region prevents radiant thermal energy from damaging the surrounding tissue.

All publications and patents mentioned in the above specification are herein incorporated by reference. Various modifications and variations of the described devices, compositions, methods, systems, and kits of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in art are intended to be within the scope of the following claims.

We claim:

1. A device comprising an elongate catheter body and a deployable procedure region, wherein the deployable procedure region is configured to deliver ablative energy to a targeted tissue region while protecting non-targeted tissue regions from thermal injury, wherein the deployable procedure region has therein an ablative region and a thermoprotective region.

2. The device of claim 1, wherein the energy is selected from the group consisting of radio-frequency energy, microwave energy, cryo-energy energy, and ultrasound energy.

3. The device of claim 1, wherein the elongate catheter body is hollow.

4. The device of claim 1, wherein the elongate catheter body is steerable.

5. The device of claim 1, wherein the elongate catheter body has thereon at least one temperature probes.

6. The device of claim 1, wherein the elongate catheter body is configured to circulate a fluid for purposes of reducing the temperature of the device.

7. The device of claim 6, wherein the fluid is saline.

8. The device of claim 1, the deployable procedure region has a shape selected from the group consisting of a balloon shape and a sail shape.

9. The device of claim 1, wherein the ablative region is designed to contact tissue targeted for ablation.

10. The device of claim 1, wherein the thermoprotective region is designed to prevent thermal injury to non-targeted tissue regions.

11. The device of claim 1, wherein the ablative region has thereon at least one electrode.

12. The device of claim 1, wherein said deployable procedure region is configured to assume a deployed position and a non-deployed position.

13. A method for ablating a tissue region, comprising
providing a device as described in claim 1, and a subject having a tissue region requiring ablation and a surrounding tissue region,
positioning the device at the tissue region, deploying the deployable procedure region such that the ablative region is in contact with the tissue region and the thermoprotective region is in contact with the surrounding tissue region, and
providing energy to the tissue region requiring ablation such that the surrounding tissue region is protected from thermal injury.

14. The method of claim 13, wherein the tissue region requiring ablation is epicardial cardiac tissue.

15. The method of claim 13, wherein the surrounding tissue region comprises esophageal tissue.

16. The method of claim 13, wherein the surrounding tissue region comprises phrenic nerve tissue.
